(19) United States
(12) Patent Application Publication
(10) Pub. No.: US 2023/0146729 A1
(43) Pub. Date: May 11, 2023

(54) ANIMAL FIBRE PRODUCT

(71) Applicant: Wool Research Organisation of New Zealand Incorporated, Shirley, Christchurch (NZ)

(72) Inventor: Robert James McClelland Kelly, Christchurch (NZ)

(21) Appl. No.: 17/910,670

(22) PCT Filed: Mar. 9, 2021
(86) PCT No.: PCT/NZ2021/050037

§ 371 Date: Sep. 9, 2022

(30) Foreign Application Priority Data
Mar. 9, 2020 (NZ) 762470

(51) Int. Cl. A61K 8/02; A23L 33/17; A61Q 5/06; B01J 20/24; B01J 20/28; C08J 5/04; D06M 11/55; D06M 11/61; D06M 11/83; D06M 15/507

(52) U.S. Cl. CPC A61K 8/027; A23L 33/17; A61Q 5/06; B01J 20/24; B01J 20/28004; C08J 5/045; D06M 11/55; D06M 11/61; D06M 11/83; D06M 15/507; B01J 2220/4856; C08J 2367/04

(57) ABSTRACT

A product having powder-like properties, the product comprising animal fibres, e.g. wool, having a length less than 10 mm, preferably less than about 3 mm, more preferably less than about 2 mm. Surprisingly, animal fibres having a length below a certain fibre length have powder-like properties, such as improved rheological properties, increased density, increased surface area and increased porosity. Also described are processes for preparing compositions and composites thereof, and uses thereof, e.g., as a filter aid, in a dietary supplement, in a filler and in a hair thickener.

### TECHNICAL FIELD

The present invention generally relates to a product comprising animal fibres. The present invention also generally relates to processes for preparing compositions and composites thereof and uses thereof.

### BACKGROUND ART

Animal fibre, particularly in the form of wool from sheep, goat or other sources, is commonly used in the manufacture of textiles. The natural fibrous structure of animal fibres lends itself well to the manufacture of a wide range of products including knitted, woven and non-woven textiles, yarns for carpet production and many others.

Animal fibre naturally has a cuticle on the outer surface. Overlapping cuticle cells give the fibre an uneven surface with regular ridges, which lead to particular properties when fibres rub against each other during processing or use. Felting of wool, due to fibres interlocking because of cuticle surface interaction, is well-known in fibre and textile processing and use; for example causing shrinkage in the laundering of woollen textiles. Known methods for addressing this problem include surface treatments and modifications, such as resin coating and decuticulation.

Wool also has a wide range of less traditional uses, including as a source of keratin protein, as a biopolymer, and for nutritional use, cosmetic use or medical use. These non-traditional uses of wool often require the native wool fibre to be processed into a powder, i.e. typically having particles with a size below 50 μm, because the natural fibrous structure is not necessary or desirable for such uses. However, processing of wool into a powder can be difficult due to the natural tendency of wool fibres to felt during any physical agitation or mixing process. This felting impedes further processing.

Processing of wool into a powder typically involves techniques that crush or pulverise the fibre, such as milling or similar techniques. Such techniques often damage the wool fibres due to heat generated during the milling process. Further, wool is not easily reduced to a powder without substantial modification of the protein properties because wool is not a brittle material under standard conditions, and brittle fracture does not commonly occur.

Accordingly, it is an object of the present invention to go some way to avoiding the above disadvantages; and/or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides a product having powder-like properties, the product comprising animal fibres having a length less than 10 mm.

In some embodiments, the product consists essentially of animal fibres having a length less than 10 mm.

In some embodiments, the product consists of animal fibres having a length less than 10 mm.

In some embodiments, the animal fibres have a length less than about 3 mm.

In some embodiments, the mean length of the animal fibres is about 3 mm to about 0.05 mm.

Preferably, the animal fibres have a length less than about 2 mm.

More preferably, the mean length of the animal fibres is about 2 mm to about 0.05 mm.

More preferably, the mean length of the animal fibres is about 2 mm to about 0.1 mm.

In some embodiments, the mean length of the animal fibres is about 2.0 mm, about 1.5 mm, about 1.0 mm, about 0.5 mm or about 0.2 mm.

In some embodiments, the animal fibres are obtained from an animal selected from the from the group consisting of a sheep, a lamb, a goat, a camel, an alpaca, a llama, a vicuna and a rabbit.

In some embodiments, the animal fibres are wool.

Preferably, the wool is sheep wool.

In some embodiments, the product is prepared by cutting animal fibres.

In some embodiments, the product has at least one powder-like property selected from the group consisting of improved rheology, increased density, increased surface area and increased porosity, compared to a product comprising longer animal fibres.

In some embodiments, the longer animal fibres have a length greater than 10 mm. In some embodiments, the longer animal fibres have a length greater than 15 mm.

In a further aspect, the present invention provides a process for preparing a product comprising animal fibres having a length less than 10 mm, the process comprising:
i) providing animal fibres having a length greater than 10 mm, and
ii) reducing the length of the animal fibres to less than 10 mm to obtain the product.

In some embodiments, the length of the animal fibres is reduced by a cutting operation.

In some embodiments, the cutting operation is performed with a rotary cutting mill or a guillotine cutter.

In some embodiments, the animal fibres have a length less than about 3 mm.

In some embodiments, the mean length of the animal fibres is about 3 mm to about 0.05 mm.

Preferably, the animal fibres have a length less than about 2 mm.

More preferably, the mean length of the animal fibres is about 2 mm to about 0.05 mm.

More preferably, the mean length of the animal fibres is about 2 mm to about 0.1 mm.

In some embodiments, the mean length of the animal fibres is about 2.0 mm, about 1.0 mm, about 0.5 mm or about 0.2 mm.

In some embodiments, the animal fibres are obtained from an animal selected from the from the group consisting of a sheep, a lamb, a goat, a camel, an alpaca, a llama, a vicuna and a rabbit.

In some embodiments, the animal fibres are wool.

Preferably, the wool is sheep wool.

In a yet further aspect, the present invention provides a product comprising animal fibres when prepared by a process of the invention.

In a still further aspect, the present invention provides a process for preparing an animal fibre composition, the process comprising:
i) providing a product of the invention, and
ii) mixing the product in a medium to obtain the animal fibre composition.

In some embodiments, the medium is a fluid.

In some embodiments, the fluid comprises a solvent.

In some embodiments, the solvent is water.

In some other embodiments, the medium is a polymeric material.

Preferably, the polymeric material is selected from the group consisting of an epoxy, a polyimide, a polylactic acid, a polyethylene, a polyacrylate, a polyurethane and a combination of any two or more thereof.

More preferably, the polymeric material is polylactic acid.

In some embodiments, the polymeric material is in the form of pellets.

In some embodiments, the polymeric material is in the form of a liquid.

In some embodiments, the animal fibre composition has improved rheology compared to a composition comprising longer animal fibres.

In some embodiments, the animal fibre composition has flow characteristics analogous to those of a powder compared to those of fibres.

In some embodiments, the product does not felt or mat when mixed in a medium.

In another aspect, the present invention provides a process for extracting a protein from animal fibres, the process comprising:
i) providing a product of the invention,
ii) subjecting the product to extraction conditions to provide an extraction solution,
iii) filtering the extraction solution to provide a filtrate and a residue, and
iv) isolating the protein from the filtrate.

In some embodiments, the extraction conditions are selected from the group consisting of oxidative sulfitolysis conditions, oxidative conditions, hydrolysis conditions and a combination of any two or more thereof.

In some embodiments, the oxidative sulfitolysis conditions comprise mixing the product with a solution comprising a copper-ammonia complex and sodium sulfite.

In some embodiments, the process comprises a further extraction step comprising:
v) suspending the residue in a solvent,
vi) filtering the suspension to obtain a second filtrate and a second residue, and
vii) isolating the protein from the second filtrate.

In some embodiments, the process comprises one, two, three, four, five or six further extraction steps.

In some embodiments, the protein is selected from the group consisting of keratin, collagen, elastin, fibroin and mixtures of any two or more thereof.

Preferably, the protein is keratin.

In another aspect, the present invention provides an animal fibre composite comprising the product of the invention and a polymeric material.

Preferably, the polymeric material is selected from the group consisting of an epoxy, a polyimide, a polylactic acid, a polyethylene, a polyacrylate, a polyurethane and a combination of any two or more thereof.

More preferably, the polymeric material is polylactic acid.

In some embodiments, the composite comprises about 5% w/w of the animal fibres relative to the polymeric material.

Preferably, the composite is substantially homogeneous.

In another aspect, the present invention provides a dietary supplement comprising the product of the invention.

In some embodiments, the dietary supplement further comprises a dietary supplement additive.

In another aspect, the present invention provides use of a product of the invention as a filter aid or absorbent material.

In another aspect, the present invention provides a process for preparing a hair thickener, the process comprising:
i) providing a product of the invention, and
ii) dyeing the animal fibres to obtain the hair thickener.

In another aspect, the present invention provides a hair thickener prepared by a process of the invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

In addition, where features or aspects of the invention are described in terms of Markush groups, those persons skilled in the art will appreciate that the invention is also thereby described in terms of any individual member or subgroup of members of the Markush group.

As used herein “(s)” following a noun means the plural and/or singular forms of the noun.

As used herein the term “and/or” means “and” or “or” or both.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “animal fibre” as used herein refers to fibres obtained from an animal, e.g. a sheep, a goat, a camel, an alpaca, a llama, a vicuna or a rabbit. The fibres may be, e.g., wool, hair, fur, or a mixture of any two or more thereof. Animal fibres are generally inert, insoluble in water, and form long protein filaments shaped like rods or wires. Proteins that may be found in animal fibres include keratin, collagen, elastin and fibroin.

The term “powder” refers to particles having a size less than 0.05 mm. The shape of the powder particles may be spherical, cubic or an irregular shape, and they typically have an aspect ratio of 1 or close to 1. Powders derived from animal fibres are commonly prepared by extracting and precipitating proteins from animal fibres, or crushing or pulverising animal fibres. Processes for converting animal fibres into a powder form often damage the fibre structure.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto and that the invention also includes embodiments of which the following description gives examples.

### DETAILED DESCRIPTION OF THE INVENTION

The present invention generally relates to a composition comprising animal fibres. The present invention also generally relates to processes for preparing compositions and composites thereof, and uses thereof, e.g., as a filter aid, in a dietary supplement, as a filler and in a hair thickener.

The present inventor has discovered that, surprisingly, animal fibres having a length below a certain fibre length, i.e. short animal fibres, have powder-like properties. Examples of powder-like properties include, but are not limited to, improved rheological properties, increased density, increased surface area and increased porosity. Advantageously, due to their powder-like properties, the short animal fibres may be suitable for uses that would ordinarily require the animal fibre to be processed into a powder. As explained above, processing the animal fibre into a powder may detrimentally affect the proteinaceous nature of the fibre.

The short animal fibres have a length of less than 10 mm, preferably less than about 3 mm, more preferably less than about 2 mm. In some embodiments, the short animal fibres have a mean length of about 3.0 mm to about 0.05 mm, about 3.0 mm to about 0.1 mm or about 3.0 mm to about 0.2 mm. Preferably, the short animal fibres have a mean length of about 2.0 mm to about 0.1 mm, about 2.0 mm to about 0.2 mm, about 1.0 mm to about 0.1 mm or about 1.0 mm to about 0.2 mm. In some embodiments, the short animal fibres have a mean length of about 3.0 mm, about 2.9 mm, about 2.8 mm, about 2.7 mm, about 2.6 mm, about 2.5 mm, about 2.4 mm, about 2.3 mm, about 2.2 mm, about 2.1 mm, about 2.0 mm, about 1.9 mm, about 1.8 mm, about 1.7 mm, about 1.6 mm, about 1.5 mm, about 1.4 mm, about 1.3 mm, about 1.2 mm, about 1.1 mm, about 1.0 mm, about 0.9 mm, about 0.8 mm, about 0.7 mm, about 0.6 mm, about 0.5 mm, about 0.4 mm, about 0.3 mm, about 0.2 mm, 0.1 mm, about 0.09 mm, about 0.08 mm, about 0.07 mm, about 0.06 mm or about 0.05 mm. Preferably, the short animal fibres have a mean length of about 2.0 mm, about 1.9 mm, about 1.8 mm, about 1.7 mm, about 1.6 mm, about 1.5 mm, about 1.4 mm, about 1.3 mm, about 1.2 mm, about 1.1 mm, about 1.0 mm, about 0.9 mm, about 0.8 mm, about 0.7 mm, about 0.6 mm, about 0.5 mm, about 0.4 mm, about 0.3 mm or about 0.2 mm. More preferably, the short animal fibres have a mean length of about 2.0 mm, about 1.5 mm, about 1.0 mm, about 0.5 mm or about 0.2 mm.

In some embodiments, the short animal fibres have a length less than about 3.0 mm, less than about 2.9 mm, less than about 2.8 mm, less than about 2.7 mm, less than about 2.6 mm, less than about 2.5 mm, less than about 2.4 mm, less than about 2.3 mm, less than about 2.2 mm, less than about 2.1 mm, less than about 2.0 mm, less than about 1.9 mm, less than about 1.8 mm, less than about 1.7 mm, less than about 1.6 mm, less than about 1.5 mm, less than about 1.4 mm, less than about 1.3 mm, less than about 1.2 mm, less than about 1.1 mm, less than about 1.0 mm, less than about 0.9 mm, less than about 0.8 mm, less than about 0.7 mm, less than about 0.6 mm, less than about 0.5 mm, less than about 0.4 mm, less than about 0.3 mm, less than about 0.2 mm, 0.1 mm, less than about 0.09 mm, less than about 0.08 mm, less than about 0.07 mm, less than about 0.06 mm or less than about 0.05 mm. Preferably, the short animal fibres have a length less than about 2.0 mm, less than about 1.9 mm, less than about 1.8 mm, less than about 1.7 mm, less than about 1.6 mm, less than about 1.5 mm, less than about 1.4 mm, less than about 1.3 mm, less than about 1.2 mm, less than about 1.1 mm, less than about 1.0 mm, less than about 0.9 mm, less than about 0.8 mm, less than about 0.7 mm, less than about 0.6 mm, less than about 0.5 mm, less than about 0.4 mm, less than about 0.3 mm or less than about 0.2 mm. More preferably, the short animal fibres have a length less than about 2.0 mm, less than about 1.5 mm, less than about 1.0 mm, less than about 0.5 mm or less than about 0.2 mm.

In some embodiments, the short animal fibres have a mean length of about 3.0 mm or less, about 2.9 mm or less, about 2.8 mm or less, about 2.7 mm or less, about 2.6 mm or less, about 2.5 mm or less, about 2.4 mm or less, about 2.3 mm or less, about 2.2 mm or less, about 2.1 mm or less, about 2.0 mm or less, about 1.9 mm or less, about 1.8 mm or less, about 1.7 mm or less, about 1.6 mm or less, about 1.5 mm or less, about 1.4 mm or less, about 1.3 mm or less, about 1.2 mm or less, about 1.1 mm or less, about 1.0 mm or less, about 0.9 mm or less, about 0.8 mm or less, about 0.7 mm or less, about 0.6 mm or less, about 0.5 mm or less, about 0.4 mm or less, about 0.3 mm or less, about 0.2 mm or less, 0.1 mm or less, about 0.09 mm or less, about 0.08 mm or less, about 0.07 mm or less, about 0.06 mm or less, or about 0.05 mm or less. Preferably, the short animal fibres have a mean length of about 2.0 mm or less, about 1.9 mm or less, about 1.8 mm or less, about 1.7 mm or less, about 1.6 mm or less, about 1.5 mm or less, about 1.4 mm or less, about 1.3 mm or less, about 1.2 mm or less, about 1.1 mm or less, about 1.0 mm or less, about 0.9 mm or less, about 0.8 mm or less, about 0.7 mm or less, about 0.6 mm or less, about 0.5 mm or less, about 0.4 mm or less, about 0.3 mm or less, or about 0.2 mm or less. More preferably, the short animal fibres have a mean length of about 2.0 mm or less, about 1.0 mm or less, about 1.5 mm or less, about 0.5 mm or less or about 0.2 mm or less.

In some embodiments, the animal fibres have a length greater than about 0.05 mm, greater than about 0.06 mm, greater than about 0.07 mm, greater than about 0.08 mm, greater than about 0.09 mm, greater than about 0.10 mm, greater than about 0.11 mm, greater than about 0.12 mm, greater than about 0.13 mm, greater than about 0.14 mm, greater than about 0.15 mm, greater than about 0.16 mm, greater than about 0.17 mm, greater than about 0.18 mm, greater than about 0.19 mm or greater than about 0.20 mm. In some embodiments, the animal fibres have a mean length of about 0.05 mm or greater, about 0.06 mm or greater, about 0.07 mm or greater, about 0.08 mm or greater, about 0.09 mm, about 0.10 mm or greater, about 0.11 mm or greater, about 0.12 mm or greater, about 0.13 mm or greater, about 0.14 mm or greater, about 0.15 mm or greater, about 0.16 mm or greater, about 0.17 mm or greater, about 0.18 mm or greater, about 0.19 mm or greater, or about 0.20 mm or greater.

In some embodiments, the present invention provides a product having powder-like properties, the product comprising animal fibres having a length of about 2 mm or less. In some embodiments, the present invention provides a product having powder-like properties, the product consisting essentially of animal fibres having a length of about 2 mm or less. In some embodiments, the present invention provides a product having powder-like properties, the product consisting of animal fibres having a length of about 2 mm or less.

In some embodiments, the present invention provides a product having powder-like properties, the product comprising animal fibres having a length of about 2.5 mm to about 0.05 mm, about 2.5 mm to about 0.1 mm, about 2.5 mm to about 0.2 mm, about 2.0 mm to about 0.05 mm, about 2.0 mm to about 0.1 mm, about 2.0 mm to about 0.2 mm, about 1.5 mm to about 0.05 mm, about 1.5 mm to about 0.1 mm, about 1.5 mm to about 0.2 mm, about 1 mm to about 0.05 mm, about 1 mm to about 0.1 mm or about 1 mm to about 0.2 mm. In some embodiments, the present invention provides a product having powder-like properties, the product consisting essentially of animal fibres having a length of about 2.5 mm to about 0.05 mm, about 2.5 mm to about 0.1 mm, about 2.5 mm to about 0.2 mm, about 2.0 mm to about 0.05 mm, about 2.0 mm to about 0.1 mm, about 2.0 mm to about 0.2 mm, about 1.5 mm to about 0.05 mm, about 1.5 mm to about 0.1 mm, about 1.5 mm to about 0.2 mm, about 1 mm to about 0.05 mm, about 1 mm to about 0.1 mm or about 1 mm to about 0.2 mm. In some embodiments, the present invention provides a product having powder-like properties, the product consisting of animal fibres having a length of about 2.5 mm to about 0.05 mm, about 2.5 mm to about 0.1 mm, about 2.5 mm to about 0.2 mm, about 2.0 mm to about 0.05 mm, about 2.0 mm to about 0.1 mm, about 2.0 mm to about 0.2 mm, about 1.5 mm to about 0.05 mm, about 1.5 mm to about 0.1 mm, about 1.5 mm to about 0.2 mm, about 1 mm to about 0.05 mm, about 1 mm to about 0.1 mm or about 1 mm to about 0.2 mm.

The animal fibre may be a fibre, e.g., wool, hair, fur, or a mixture of any two or more thereof, obtained from an animal. Suitable animals include, but are not limited to, sheep, lambs, goats, camels, alpacas, llamas, vicunas and rabbits. Preferably, the animal fibre is a wool, e.g., sheep wool or goat wool. More preferably, the animal fibre is sheep wool. Although the present invention is described with reference to particular animal fibres, the invention is particularly useful for any animal fibre having a cuticle.

The length of the animal fibres may be reduced by conventional methods. Preferably, the length of the animal fibres is reduced by a cutting operation. The cutting operation may be performed with, e.g., a rotary cutting mill or a guillotine cutter. Advantageously, the use of a cutting operation to reduce the length of the animal fibres avoids the need for crushing or pulverising techniques. As a result, damage to the animal fibre structure may be minimised.

Fibres having improved rheological properties refers to fibres having increased flowability relative to longer animal fibres. For example, the short animal fibres may have increased flowability when the fibres are mixed or agitated in a medium such as a fluid, particularly a liquid. Surprisingly, animal fibres below a certain length, particularly animal fibres having a length less than about 2 mm, may exhibit flow characteristics analogous to the flow characteristics of a powder, compared with those of fibres.

One cause of reduced flowability in animal fibres is felting or matting. In some embodiments, mixing or agitating the short animal fibres in a medium leads to reduced felting and/or matting compared with longer animal fibres. Advantageously, reduced felting allows for more efficient mixing of the fibres in a medium. In some embodiments, mixing or agitating the short animal fibres in a medium leads to no felting or matting.

Fibres having improved rheological properties may be beneficial in any process in which it is necessary or preferable for the fibres be free flowing. For example, a process that involves mixing or agitating animal fibres in a medium, such as a process for extracting a protein from animal fibres. Examples of proteins that may be extracted from short animal fibres include, but are not limited to, keratin, collagen, elastin, fibroin and mixtures of any two or more thereof. Preferably, keratin is extracted from the short animal fibres.

The extraction process may be performed by subjecting a composition comprising short animal fibres to extraction conditions, such as oxidative sulfitolysis conditions, oxidative conditions, hydrolysis conditions or a combination of any two or more thereof. Conventional extraction conditions known in the art are applicable in the present invention. The extraction conditions typically involve mixing the animal fibres in a suitable solvent, such as a solvent comprising water, under conditions that cause at least a portion of at least one protein to be extracted into the solvent. The extraction solution may then be filtered to provide a filtrate and a residue. The protein is typically isolated from the filtrate, e.g. by precipitation.

Generally, further protein may be extracted from the residue by suspending the residue in a suitable solvent, such as a solvent comprising water, for a period of time with or without mixing or agitation. The solution may then be filtered to obtain a second filtrate and a second residue. Further protein may be isolated from the second filtrate, e.g. by precipitation. The second residue may be further extracted by the same process, and so on, until no further protein is isolated from the filtrate. For example, in some embodiments, the extraction process may comprise one, two, three, four, five or six further extraction steps.

Advantageously, performing the extraction with a composition comprising short animal fibres may provide more efficient mixing and improve the efficiency of the extraction process. Without wishing to be bound by theory, it is believed that the increased surface area and/or increased porosity of the short animal fibres may also improve the efficiency of the extraction process. By improving the efficiency of the process, the yield of the protein from each extraction step may be increased. As a result, the overall yield of the protein may be increased and/or fewer extraction steps may be required.

Improved rheological properties may also be beneficial in processes that involve forming animal fibre composites comprising short animal fibres and one or more additional components. Advantageously, the powder-like properties of the short animal fibres facilitate mixing of the fibres and the additional component(s). Consequently, it is possible to obtain a composite comprising a substantially homogeneous mixture of the short animal fibres and the additional component(s).

In some embodiments, the animal fibre composite comprises short animal fibres and a polymeric material. The composite comprising short animal fibres and a polymeric material may be useful, e.g., as a filler material.

Examples of suitable polymeric materials include, but are not limited to, an epoxy, a polyimide, a polylactic acid, a polyethylene, a polyacrylate, a polyurethane and a combination of any two or more thereof. In some embodiments, the polymeric material is a thermoplastic material. In some embodiments, the polymeric material is a low melt temperature polymeric material. Preferably, the polymeric material is a polylactic acid.

In some embodiments, the animal fibre composite is prepared by mixing the short animal fibres with pellets of a polymeric material to form a blend. The blend is melted and optionally compressed to form the animal fibre composite. In some other embodiments, the animal fibre composite is prepared by mixing the short cut fibres with a melted polymeric material to provide a blended mixture of short animal fibres and melted polymeric material. The blended mixture may be extruded to form an animal fibre composite of a desired shape.

The short animal fibres may have a higher bulk density relative to longer fibres. For example, the short animal fibres may have a bulk density of about 500 kg/m³. For comparison, loose wool typically has a bulk density of about 30 kg/m³. Animal fibres having a high bulk density are useful in a variety of applications, such as in a dietary supplement.

In some embodiments, the dietary supplement comprises short animal fibres. The dietary supplement may further comprise one or more dietary supplement additives. Any conventional dietary supplement additive known in the art may be used. For example, one or more dietary supplement additives may be selected from the group consisting of extenders, fillers, lubricants, preservatives, diluents, disintegrating agents, and flavouring agents. The dietary supplement may be provided in any form suitable for consumption. Examples of suitable forms include, but are not limited to, capsules, pills, tablets, powder-like forms, granules and gels. Preferably, the dietary supplement is provided in the form of capsules.

Other applications in which the short animal fibres may be useful include as a filter aid and an absorbent material, e.g. for absorbing gases, oil and/or moisture.

Advantageously, the short fibres may retain certain fibre-like properties. For example, in some embodiments the short animal fibres may hold a static charge. Without wishing to be bound by theory, it is believed the ability to hold static charge is due to the proteinaceous nature of the fibres.

The following non-limiting examples are provided to illustrate the present invention and in no way limit the scope thereof.

### EXAMPLES

1. Processing Short Cut Wool Compared with Loose Wool

Loose scoured wool was combined with water at a ratio of 1:20 w/w and stirred using an overhead blade stirrer. The loose wool had a staple length of approximately 5 cm. The wool moved as one material in the solution. Without wishing to be bound by theory, it is believed close fibre interactions held the fibres together and caused felting of the fibres. The felting of fibres led to poor mixing of the solution with the wool and poor penetration of the water into the fibre network.

A similar mass of wool was cut to a fibre length of approximately 15 mm and mixed with water at a ratio of 1:20. When mixed with an overhead blade stirrer the wool again formed a fibrous mat resulting in poor mixing, etc.

Wool chopped to a fibre length of 3 mm behaved differently to the fibres described above. When stirred in a similar manner, initially, the wool did not move as one matted material and superior mixing was achieved. However, felting occurred after extended stirring. The felted wool balls that formed were poorly penetrated by the water and, as a result, the mixture was not homogeneous.

Wool cut to a fibre length of 2 mm was mixed with water at a ratio of 1:20 and stirred using an overhead blade stirrer. Full mixing readily occurred and the mixture was homogenous. No felting or matting of the fibres occurred after 24 hours of stirring.

Several fibre lengths below 2 mm were evaluated, including 1 mm and 0.2 mm fibres. All fibres below 2 mm in length exhibited powder-like properties, e.g. did not felt or matt, when stirred in water.

2. Keratin Extraction from Wool

Soluble keratin proteins were extracted from two samples of wool using an oxidative sulfitolysis process as described in U.S. Pat. No. 7,148,327 (the entire contents of which is incorporated by reference herein). The first sample comprised 90 g of wool cut to 10 mm length. The second sample comprised 90 g of wool cut to 0.2 mm length. Each sample was immersed in 550 ml of copper—ammonia complex, sodium sulfite added and the mixture made up to 1 L. Only gentle oscillating agitation was achieved with the 10 mm wool sample due to the tendency of the wool to felt and then restrict mixing. Stirred agitation was achieved with the 0.2 mm wool sample. Each solution was filtered and the filtrate replaced with an equivalent volume of water every 24 hours for 5 days. Following extraction, the soluble protein was precipitated from each filtrate aliquot. The total yield of soluble protein from both samples was about 34%.

The results of these experiments demonstrate that a significant improvement in extraction time was achieved with the 0.2 mm fibre sample. Specifically, an equivalent protein extraction was achieved in 3 days with the 0.2 mm fibre sample compared to 5 days with the 10 mm fibre sample.

| | Yield of protein | Yield of protein |
|---|---|---|
| | extracted from | extracted from |
| | solution using 10 | solution using 0.2 |
| | mm fibre | mm fibre |
| Copper ammonia solution | 0 | 9.4 |
| Water solution 1 | 11.9 | 15.9 |
| Water solution 2 | 10.2 | 8.7 |
| Water solution 3 | 7.2 | 0 |
| Water solution 4 | 5.1 | 0 |
| Total | 34.4 | 34.0 |

Similar improvements in extraction efficiency were also observed when extraction processes including oxidation and hydrolysis were employed.

### 3. Preparation of a Dietary Supplement Powder

Loose wool (bulk density approximately 30 kg/m³) was cut to a length of 0.2 mm. The bulk density of the cut wool was 470 kg/m³. The higher density permitted a combination of 200 mg of the cut wool with 300 mg of a common dietary supplement additive (starch) to be filled into a capsule to provide a dietary supplement.

### 4. Preparation of a Filler in Composites

Wool cut to a length of 0.2 mm was combined with a low melt temperature polymer, polylactic acid (PLA). Two different methods of mixing were tested. In the first method, 0.2 mm cut wool was combined at a ratio of 5% w/w with PLA pellets and the mixture stirred to create a blend. The blend was melted and compressed to create a composite of cut wool embedded in PLA. In the second method, PLA was raised above its melt temperature and 0.2 mm cut wool added to the resulting liquid. This mixture was blended as a liquid to ensure a homogeneous mixture and then extruded into desired shapes to create a composite PLA/wool material.

Neither method of mixing was effective when performed with animal fibres that were not cut to a length according to the present invention. As a result, homogeneous composite materials could not be obtained.

### 5. Comparison of Cut Wool and Activated Carbon as a Filter Aid

Wool cut to a length of 0.2 mm was found to be effective at removing pollutant gases in comparison to acid impregnated activated carbon on a per mass and per volume basis. Loose wool (bulk density approximately 30 kg/m³) was cut to a length of 0.2 mm. The cut wool had a bulk density of 470 kg/m³. Gas absorption properties of the 0.2 mm cut wool were compared to those of acid impregnated activated carbon (bulk density of 520 kg/m³); a filtration medium. 5 g of each material was placed in a chamber and the chamber filled with an equivalent volume of sulfur dioxide. After 1500 seconds a similar amount of sulfur dioxide had been absorbed by each material, specifically, 85% for 0.2 mm cut wool fibre compared to 93% for the acid impregnated activated carbon.

### 6. Preparation of a Hair Thickener

Wool was cut to 1 mm length and dyed brown using conventional dye processes. The resulting dyed cut wool had several advantageous properties. It was coloured the same as human hair, flowable like a powder, and able to hold static charge. Without wishing to be bound by theory, it is believed the ability to hold a static charge is due to the proteinaceous nature of the fibre. Due to this combination of features the brown, short cut wool powder was useful as a topical hair thickening product. When topically applied to the head, the short cut fibres electrostatically attached to the hair to give cosmetic the appearance of greater thickness.

It is not the intention to limit the scope of the invention to the abovementioned examples only. As would be appreciated by a skilled person in the art, many variations are possible without departing from the scope of the invention as set out in the accompanying claims.

1. A product having powder-like properties, the product comprising animal fibres having a length less than 10 mm.

2. The product of claim 1, wherein the mean length of the animal fibres is about 3 mm to about 0.05 mm.

3. The product of claim 1 or 2, wherein the mean length of the animal fibres is about 2 mm to about 0.1 mm.

4. The product of any one of claims 1-3, wherein the animal fibres are wool.

5. The product of claim 4, wherein the wool is sheep wool.

6. The product of any one of claims 1-5, wherein the product is prepared by cutting animal fibres.

7. The product of any one of claims 1-6, wherein the product has at least one powder-like property selected from the group consisting of improved rheology, increased density, increased surface area and increased porosity, compared to a product comprising animal fibres having a length greater than 10 mm.

8. A process for preparing a product comprising animal fibres having a length less than 10 mm, the process comprising:
i) providing animal fibres having a length greater than 10 mm, and
ii) reducing the length of the animal fibres to less than 10 mm to obtain the product.

9. The process of claim 8, wherein the length of the animal fibres is reduced by a cutting operation.

10. The process of claim 9, wherein the cutting operation is performed with a rotary cutting mill or a guillotine cutter.

11. The process of any one of claims 8-10, wherein the mean length of the animal fibres is reduced to about 3 mm to about 0.05 mm.

12. The process of any one of claims 8-11, wherein the mean length of the animal fibres is reduced to about 2 mm to about 0.1 mm.

13. The process of any one of claims 8-12, wherein the animal fibres are wool.

14. The process of claim 13, wherein the wool is sheep wool.

15. A product comprising animal fibres when prepared by the process of any one of claims 8-14.

16. A process for preparing an animal fibre composition, the process comprising:
i) providing a product of any one of claims 1-7 and 15, and
ii) mixing the product in a medium to obtain the animal fibre composition.

17. The process of claim 16, wherein the medium is a fluid.

18. The process of claim 17, wherein the fluid comprises water.

19. The process of claim 16, wherein the medium is a polymeric material.

20. The process of claim 19, wherein the polymeric material is polylactic acid.

21. The process of claim 19 or 20, wherein the polymeric material is in the form of pellets.

22. The process of claim 19 or 20, wherein the polymeric material is in the form of a liquid.

23. The process of any one of claims 16-22, wherein the animal fibre composition has improved rheology compared to a composition comprising animal fibres having a length greater than 10 mm.

24. A process for extracting a protein from animal fibres, the process comprising:
i) providing a product of any one of claims 1-7 and 15,
ii) subjecting the product to extraction conditions to provide an extraction solution,
iii) filtering the extraction solution to provide a filtrate and a residue, and
iv) isolating the protein from the filtrate.

25. The process of claim 24, wherein the extraction conditions are selected from the group consisting of oxidative sulfitolysis conditions, oxidative conditions, hydrolysis conditions and a combination of any two or more thereof.

26. The process of claim 25, wherein the oxidative sulfitolysis conditions comprise mixing the product with a solution comprising a copper—ammonia complex and sodium sulfite.

27. The process of any one of claims 24-26, wherein the process comprises a further extraction step comprising:
v) suspending the residue in a solvent,
vi) filtering the suspension to obtain a second filtrate and a second residue, and
vii) isolating the protein from the second filtrate.

28. The process of claim 27, wherein the process comprises one, two, three, four, five or six further extraction steps.

29. The process of any one of claims 24-28, wherein the protein is selected from the group consisting of keratin, collagen, elastin, fibroin and mixtures of any two or more thereof.

30. An animal fibre composite comprising the product of any one of claims 1-7 and 15 and a polymeric material.

31. The animal fibre composite of claim 30, wherein the polymeric material is polylactic acid.

32. The animal fibre composite of claim 30 or 31, wherein the composite comprises about 5% w/w of the animal fibres relative to the polymeric material.

33. The animal fibre composite of any one of claims 30-32, wherein the composite is substantially homogeneous.

34. A dietary supplement comprising the product of any one of claims 1-7 and 15.

35. The dietary supplement of claim 34, wherein the dietary supplement further comprises a dietary supplement additive.

36. Use of a product of any one of claims 1-7 and 15 as a filter aid or absorbent material.

37. A process for preparing a hair thickener, the process comprising:
i) providing a product of any one of claims 1-7 and 15, and
ii) dyeing the animal fibres to obtain the hair thickener.

38. A hair thickener when prepared by the process of claim 37.
